(19) United States
(12) Patent Application Publication
(10) Pub. No.: US 2017/0240960 A1
(43) Pub. Date: Aug. 24, 2017

(54) SPHERICAL NUCLEIC ACIDS (SNA) FLARE BASED FLUORESCENCE IN SITU HYBRIDIZATION

(71) Applicant: AuraSense, LLC, Skokie, IL (US)

(72) Inventors: David A. Giljohann, Chicago, IL (US); Richard Kang, Wilmette, IL (US); Pinal Patel, Skokie, IL (US)

(73) Assignee: AuraSense, LLC, Skokie, IL (US)

(21) Appl. No.: 15/437,524

(22) Filed: Feb. 21, 2017

Related U.S. Application Data
(60) Provisional application No. 62/299,454, filed on Feb. 24, 2016.

(51) Int. Cl. C12Q 1/68

(52) U.S. Cl. CPC C12Q 1/6841; C12Q 1/682

(57) ABSTRACT

The invention relates to a method of performing in situ hybridization such as fluorescence in situ hybridization (FISH) using liposomal spherical nucleic acids (L-SNAs) nanoparticles labeled with dye molecules. The nanoparticles contain one or more nucleic acids that recognize a target of interest in a sample.

### RELATED APPLICATIONS

This application claims the benefit under 35 U.S.C. §119 of U.S. provisional application 62/299,454, filed Feb. 24, 2016 the entire contents of which is incorporated herein by reference.

### FIELD OF INVENTION

The present invention generally relates to methods of using nanoparticles containing oligonucleotides labeled with dye molecules to detect a target nucleic acid, and kits and compositions thereof.

### BACKGROUND

Fluorescence in situ hybridization (FISH) for the detection of mRNA is a widely used technique to image mRNA molecules in cells, or tissue sections, that are fixed, then permeabilized to allow probe access to the gene target. Current technology utilizes multiple fluorescently labeled oligonucleotide probes with unique sequences to recognize a single mRNA target. After the probes are hybridized to the target, the samples can be imaged using fluorescence microscopy. The maximum potential signal intensity using individually fluorescently labeled oligonucleotide probes is directly related to the number of unique sequences used against a single gene target. In order to increase the fluorescence intensity for each mRNA molecule, more unique oligonucleotide sequences must be added to the probe set increasing cost and complexity.

Other methods of FISH utilize a signal amplification method by using multiple stages of hybridization of branched oligonucleotides to a set of multiple unique target probes. After multiple rounds of hybridization of branched oligonucleotides, fluorophores are hybridized resulting in a multiplication of signal per probe. These current techniques rely on a multiple steps and/or oligonucleotide sequences to produce an amplification of target signal.

### SUMMARY OF THE INVENTION

In some aspects, the invention is a method of detecting a target nucleic acid in a sample by in situ hybridization, by incubating a sample with a liposomal spherical nucleic acid (L-SNA) probe and detecting probe hybridized to a nucleic acid target in the sample by visualization of a signal emitted from the L-SNA probe. In some embodiments the in situ hybridization is fluorescence in situ hybridization (FISH). In other embodiments the sample is fixed.

The L-SNA probe, in some embodiments, is comprised of a liposomal core and an oligonucleotide shell or 2-500 oligonucleotides surrounding the liposomal core and linked to a dye molecule or a plurality of dye molecules. The dye molecule or a plurality of dye molecules may be attached to either the 5′- or 3′-end of the oligonucleotides. In some embodiments the dye molecule is a fluorophore. The signal depicting the nucleic acid target, in some embodiments, is visualized by fluorescence microscopy.

In other embodiments the oligonucleotides are anchored to the liposomal core and point radially outwards from the core. In yet other embodiments the oligonucleotides are attached to a hydrophobic anchor group at the 5′- or 3′-end to insert the oligonucleotides into the liposomal core and/or anchor the oligonucleotides to the liposomal core. The hydrophobic anchor group may be a lipid. The lipid may be a di-stearyl group.

In some embodiments the liposomal core comprises 1,2-dioleoyl-sn-glycero-3-phosphatidylcholine (DOPC).

The L-SNA is comprised of about 100 oligonucleotides in other embodiments. In yet other embodiments the L-SNA is comprised of 80-120 oligonucleotides or 50-200 oligonucleotides.

The sample in some embodiments is a cell or a tissue. In other embodiments the sample is a low abundance cell.

In some embodiments the method is used to diagnose a disease.

The nucleic acid target may be an RNA or a DNA. In some embodiments the oligonucleotide is DNA or RNA.

The method may also comprise the following methods: attaching the sample, wherein the sample is a cell, to a solid surface; permeabilizing the cell in 70% ethanol for at least 1 hour and up to a week at 2-8 ° C.; incubating the cell with L-SNA probes in hybridization buffer composed of 1:1:1:7 ratio of dextran sulfate, formamide, 20×SSC, and nuclease free water in the dark at 37° C. for 4-16 hours; counterstaining the cell with a nuclear stain; and detecting a fluorescent signal.

In other aspects the invention is compositions and kits for performing the methods described herein. A kit may include a lipid for producing a liposome, a fluorescent dye or plurality of fluorescent dyes, one or more reagents for performing a FISH assay, and instructions for performing a FISH assay using a L-SNA probe. In some embodiments, the kit may include liposomes for forming a liposomal core or a metal for forming a solid core.

In some embodiments the kit further includes a set of oligonucleotides for preparing an L-SNA probe. In some embodiments the oligonucleotide is DNA or RNA. In other embodiments the fluorescent dye is linked to the oligonucleotides. The fluorescent dye may be attached to either the 5′- or 3′-end of the oligonucleotide. In some embodiments the fluorescent dye is a fluorophore. In other embodiments the fluorophore is Cy5.

In some embodiments the oligonucleotides have a hydrophobic anchor group at a 5′ or 3′ end. The hydrophobic anchor group is a di-stearyl group. In other embodiments the lipid is 1,2-dioleoyl-sn-glycero-3-phosphatidylcholine (DOPC).

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A-1B. General structure of labeled probes. (A) Binding of oligonucleotides (oligos) labeled with a dye to a complementary sequence on a nucleic acid. (B) General structure of L-SNA probes for fluorescence in situ hybridization (FISH), wherein one end of the oligonucleotide is linked to a dye facing outward from the core and the opposite end of the oligonucleotide is linked to a hydrophobic group that anchors the oligonucleotide to the liposomal core.

FIG. 2. Comparison of fluorescence in situ hybridization (FISH) L-SNA probes and commercially available FISH probes in adherent cells. Adherent cells grown on coverslips are fixed, permeabilized and subsequently incubated with SNAs containing ACTB control oligonucleotides, SNAs containing oligonucleotides that bind ACTB ((β-Actin) mRNA transcripts, or the commercially available Stellaris probes that bind to ACTB ((β-Actin) mRNA transcripts for comparison. Cells are also treated with DAPI to visualize the nucleus. Cells are imaged with a 40× objective using a wide-field fluorescent microscope. Same scale settings are applied to all sample images taken. Using ImageJ, the total cell fluorescence is analyzed for each cell area in each sample and relative fluorescence compared between treatments.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Methods of using liposomal spherical nucleic acids (L-SNA), in which the nucleic acids are labeled with a dye or a plurality of dyes, to detect nucleic acid targets in situ are provided herein. In some aspects the method involves using labeled L-SNA as probes for fluorescence in situ hybridization (FISH) to detect a nucleic acid target of interest. Each L-SNA FISH probe is arrayed with many dye molecules and utilizes one or more unique oligonucleotide sequences to recognize its gene target. A single binding event of an L-SNA molecule to its target provides high signal amplification due to the density of fluorophore molecules on each particle. This high ratio of fluorophore to probe increases the detectable range for targets of lower abundance.

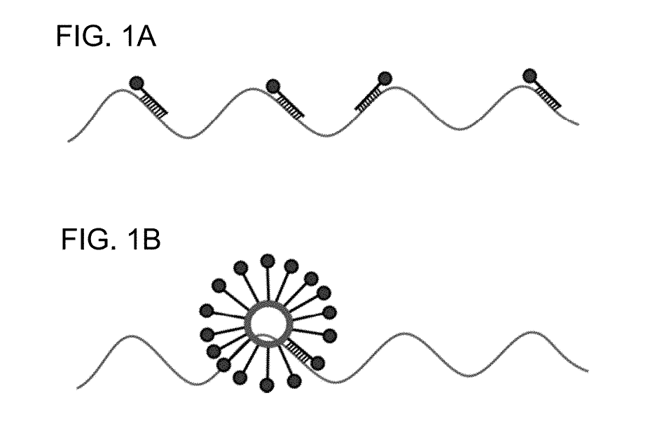

The invention herein, in some aspects, describes a method of performing fluorescence in situ hybridization (FISH) using L-SNA, in which the nucleic acids are labeled with a dye or a plurality of dyes, to detect a complementary nucleic acid target of interest. Quite surprisingly, the L-SNA FISH probes have better performance than a currently available commercial FISH system that implements multiple probes toward a single gene target. Each L-SNA FISH probe is arrayed with multiple dye molecules and utilizes one or more unique oligonucleotide sequences to recognize its gene target. A single binding event of an L-SNA molecule to its target provides high signal amplification due to the density of fluorophore molecules on each particle (FIG. 1). The high ratio of fluorophore to probe increases the detectable range for targets, providing an advantage that can be particularly important when attempting to detect targets of lower cellular abundance.

Typically, FISH methods involve the following steps: (a) providing a sample fixed to a solid support; (b) treatment of the sample to increase accessibility of probe nucleic acid to target nucleic acid, e.g., with chemical or protease treatments (e.g., 10 mM citrate buffer pH 6.0 with 8% sodium thiocyanate; 0.2N HCl; or proteinase K at 25 μg/ml or pepsin at 750 U/ml), (c) contacting the tissue or material containing the target nucleic acid with labeled probes of a probe set to form specific hybridization complexes, (d) post hybridization washes of the complexes to selectively remove probes that are not specifically hybridized to the target, and (e) detection of FISH signals from probes that have formed hybridization complexes with target nucleic acids. Such methods are described in a number of sources, including: Gall and Pardue, Methods of Enzymology 1981; 21:470-480; Henderson, International Review of Cytology, 1982; 76:1-46; Angerer, et al., in Genetic Engineering: Principles and Methods (Setlow and Hollaender, Eds.) 1985; vol. 7, pp. 43-65, Plenum Press, New York; and Varella-Garcia M et al., EGFR fluorescence in situ hybridisation assay: guidelines for application to non-small-cell lung cancer, J. Olin. Pathol. 2009; 62:970-977.

Fluorescence in situ hybridization (FISH), the assay of choice for localization of specific nucleic acids sequences in native context, is a 20-year-old technology that has developed continuously. Over its maturation, various methodologies and modifications have been introduced to optimize the detection of DNA and RNA. The pervasiveness of this technique is largely because of its wide variety of applications and the relative ease of implementation and performance of in situ studies. The development of in situ technologies provides a wealth of information regarding the locations and expression patterns of genes in single cells, and insight into the correlation of gene expression patterns with particular cellular phenotypes. This is particularly important in studies of development and disease progression, where complicated, finely demarcated gene expression programs are in play.

In situ hybridization assays are well known and are generally described in Angerer et al., Methods Enzymol. 152:649-660 (1987). In an in situ hybridization assay, cells, e.g., from a tissue sample or biopsy, are fixed to a solid support, typically a glass slide. If DNA is to be probed, the cells are denatured with heat or alkali. The cells are then contacted with a hybridization solution at a moderate temperature to permit annealing of specific probes that are labeled. These methods can be adapted to use with the L-SNA probes described herein. The probes are preferably labeled with radioisotopes or fluorescent reporters. FISH uses fluorescent probes.

FISH is a cytogenetic technique used to detect and localize specific polynucleotide sequences in cells. For example, FISH can be used to detect DNA sequences on chromosomes. FISH can also be used to detect and localize specific RNA targets (mRNA, lncRNA and miRNA) in cells, circulating tumor cells, and tissue samples. In this context, it can help define the spatial-temporal patterns of gene expression within cells and tissues. Fluorescence microscopy can be used to find out whether and where the fluorescent probes are bound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

Cells, circulating tumor cells (CTCs), or formalin-fixed paraffin-embedded (FFPE) or frozen tissue sections may be fixed, then permeabilized to allow target accessibility. FISH may also be done on unfixed cells. The nanoparticles of this invention are typically composed of a liposomal core, a shell of oligonucleotides, a dye and a hydrophobic or lipid anchor group. The liposomal core is formed by arranging lipids into a small hollow liposome; the shell of oligonucleotides is formed by arranging oligonucleotides pointing radially outwards from the core; the dye, which includes, but it is not limited to a fluorophore or a plurality of fluorophores (e.g. Cy5), is attached to either the 5′- or 3′-end of each oligonucleotide in the oligonucleotide shell, depending on whether the oligonucleotides are arranged with the 5′- or 3′-end facing outward from the core; and a hydrophobic (e.g. lipid) anchor group attached to either the 5′- or 3′-end of the oligonucleotide, depending on whether the oligonucleotides are arranged with the 5′- or 3′-end facing outward from the core. In some instances, the liposomal core may contain one or more fluorophores or dye molecule(s) to further increase the signal emitted by the L-SNAs. The anchor acts to both drive insertion into the liposome and to anchor the oligonucleotides to the liposomal core.

Thus, the invention involves methods for using L-SNA molecule to perform FISH imaging. A single binding event of an L-SNA molecule to its target provides high signal amplification due to the density of fluorophore molecules on each oligonucleotide. This high ratio of fluorophore to probe increases the detectable range for targets of lower abundance.

A liposome is a structure composed of at least one lipid layer membrane that encloses an internal compartment. Liposomes may be characterized according to the membrane type and size. Small unilamellar vesicles (SUVs) have a single membrane and typically range between 0.02 and 0.05 pm in diameter; large unilamellar vesicles (LUVs) are typically larger than 0.05 pm. Oligolamellar large vesicles and multilamellar vesicles have multiple, usually concentric, membrane layers and are typically larger than 0.1 pm. Liposomes with several nonconcentric membranes, i.e., several smaller vesicles contained within a larger vesicle, are termed multivesicular vesicles.

Other lipids may be included in the lipid nanoparticle for a variety of purposes, such as to prevent lipid oxidation or to attach ligands onto lipid nanoparticle surface. Any of a number of lipids may be present, including amphipathic, neutral, cationic, and anionic lipids. Such lipids can be used alone or in combination. Additional components that may be present in a lipid nanoparticle include bilayer stabilizing components such as polyamide oligomers, peptides, proteins, detergents, lipid-derivatives, such as PEG coupled to phosphatidylethanolamine and PEG conjugated to ceramides. The lipid nanoparticles may also include one or more of a second amino lipid or cationic lipid, a neutral lipid, a sterol, and a lipid selected to reduce aggregation of lipid particles during formation, which may result from steric stabilization of particles which prevents charge-induced aggregation during formation. As used herein, the term “amino lipid” is meant to include those lipids having one or two fatty acid or fatty alkyl chains and an amino head group (including an alkylamino or dialkylamino group) that may be protonated to form a cationic lipid at physiological pH.

The nanostructure of the invention includes a core. The core may be a solid or a hollow core, such as a liposomal core. A solid core is a spherical shaped material that does not have a hollow center. The term spherical as used herein refers to a general shape and does not imply or is not limited to a perfect sphere or round shape. It may include imperfections.

Solid cores can be constructed from a wide variety of materials known to those skilled in the art including but not limited to: noble metals (gold, silver), transition metals (iron, cobalt) and metal oxides (silica). In addition, these cores may be inert, paramagnetic, or supramagnetic. These solid cores can be constructed from either pure compositions of described materials, or in combinations of mixtures of any number of materials, or in layered compositions of materials. In addition, solid cores can be composed of a polymeric core such as amphiphilic block copolymers, hydrophobic polymers such as polystyrene, poly(lactic acid), poly(lactic co-glycolic acid), poly(glycolic acid), poly(caprolactone) and other biocompatible polymers known to those skilled in the art.

A nanostructure core may be formed from any suitable material. For instance, in one embodiment, a nanostructure core comprises an inorganic material. The inorganic material may include, for example, a metal (e.g., Ag, Au, Pt, Fe, Cr, Co, Ni, Cu, Zn, Ti, Pd and other metals), a semiconductor (e.g., Rh, Ge, silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 wt %, 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen. In certain embodiments, the nanostructure core does not include a polymeric material (e.g., it is non-polymeric).

The core may alternatively be a hollow core, which has at least some space in the center region of a shell material. Hollow cores include liposomal cores.

A liposomal core as used herein refers to a centrally located core compartment formed by a component of the lipids or phospholipids that form a lipid bilayer. “Liposomes” are artificial, self-closed vesicular structure of various sizes and structures, where one or several membranes encapsulate an aqueous core. Most typically liposome membranes are formed from lipid bilayers membranes, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agent.

“Lipid” refers to its conventional sense as a generic term encompassing fats, lipids, alcohol-ether-soluble constituents of protoplasm, which are insoluble in water. Lipids usually consist of a hydrophilic and a hydrophobic moiety. In water lipids can self-organize to form bilayers membranes, where the hydrophilic moieties (head groups) are oriented towards the aqueous phase, and the lipophilic moieties (acyl chains) are embedded in the bilayers core. Lipids can comprise as well two hydrophilic moieties (bola amphiphiles). In that case, membranes may be formed from a single lipid layer, and not a bilayer. Typical examples for lipids in the current context are fats, fatty oils, essential oils, waxes, steroid, sterols, phospholipids, glycolipids, sulpholipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl, phosphatidylcholines and phosphatidylethanolamines and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g. lysolecithins. Mixed formulations are also possible, particularly when the non-cationic lipids are derived from natural sources, such as lecithins (phosphatidylcholines) purified from egg yolk, bovine heart, brain, liver or soybean.

The liposomal core can be constructed from a wide variety of lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In some instances, the liposomal core may contain one or more dye molecules to further increase the signal emitted by the L-SNAs.

The lipid nanoparticle may include a cationic lipid. The cationic lipid may be, for example, N,N-dioleyl-N,N-dimethylammonium chloride (DODAC), N,N-distearyl-N,N-dimethylammonium bromide (DDAB), N-(1-(2,3 -dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3 -morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoy1-3 -trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3 -(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d] [1,3] dioxol-5-amine, (6Z,9Z,28Z,31Z)-heptatriaconta-6,9,28,31-tetraen-19-yl-4-(dimethylamino)butanoate, or a mixture thereof.

Other cationic lipids, which carry a net positive charge at about physiological pH, in addition to those specifically described above, may also be included in the lipid nanoparticle. Such cationic lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3.beta.-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), and 1,2-dioleoyl-sn-glycero-3-phosphocholine (“DOPC”).

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine, monophosphoryl lipid A (MPLA), or glycopyranoside lipid A (GLA).

The liposome is formulated in an aqueous solution. An aqueous solution is a water-based solution. Aqueous solutions are well known to the skilled artisan.

The liposomal aqueous based solution is mixed with an organic solvent that contains the hydrophobic agent. An organic solvent is a carbon based solution that is capable of dissolving another substance and is miscible in an aqueous solution. Organic solvents include but are not limited to benzene, toluene, xylene, tetrahydrofurane, methyltetrahydrofurane, N,N-dimethylformamide, acetone, acetonitrile, anisole, dichloromethane, dimethylsulfoxide (DMSO), chlorobenzene, 1,2-dichlorobenzene and mixtures thereof.

The organic solvent is then removed from the liposomal solution using any technique known in the art. One example of a useful technique is diafiltration using tangential-flow filtration (TFF).

A diagnostic oligonucleotide is an oligonucleotide that interacts with a cellular marker to identify the presence of the marker in a cell or subject. Diagnostic oligonucleotides are well known in the art and typically include a label or are otherwise detectable.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis).

An oligonucleotide of the nanostructure can be single stranded or double stranded. A double stranded oligonucleotide is also referred to herein as a duplex. Double-stranded oligonucleotides of the invention can comprise two separate complementary nucleic acid strands.

The nucleic acids useful in the nanostructures of the invention are synthetic or isolated nucleic acids.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded oligonucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

Oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH₂, NHR, NR₂,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′- methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C₁-C₆ for straight chain, C₃-C₆ for branched chain), and more preferably 4 or fewer Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. The term “alkenyl” includes unsaturated aliphatic groups analogous in length and possible substitution to the alkyls described above, but that contain at least one double bond. The term alkenyl includes both “unsubstituted alkenyls” and “substituted alkenyls,” the latter of which refers to alkenyl moieties having independently selected substituents replacing a hydrogen on one or more carbons of the hydrocarbon backbone.

The term “hydrophobic modifications’ refers to modification of bases such that overall hydrophobicity is increased and the base is still capable of forming close to regular Watson—Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The oligonucleotide shell can be constructed from a wide variety of nucleic acids including, but not limited to: single-stranded deoxyribonucleotides, ribonucleotides, and other single-stranded oligonucleotides incorporating one or a multiplicity of modifications known to those in the art, double-stranded deoxyribonucleotides, ribonucleotides, and other double-stranded oligonucleotides incorporating one or a multiplicity of modifications known to those in the art, oligonucleotide triplexes incorporating deoxyribonucleotides, ribonucleotides, or oligonucleotides that incorporate one or a multiplicity of modifications known to those in the art.

The nanostructures of the invention contemplate the use of linkers. The linkers may be linkers between the hydrophobic molecule and other therapeutic or diagnostic molecules. The linkers may also be nucleic acid linkers between nucleic acids, including standard phosphodiester internucleotide linkages as well as modified internucleotide linkages. The linkers may also be non-standard linkages that link hydrophobic molecules with nucleic acids or with other compounds such as proteins. As used herein, the term nucleotide linkage includes a naturally occurring, unmodified phosphodiester moiety (—O—(PO²⁻)—O—) that covalently couples adjacent nucleomonomers as well as any analog or derivative of the native phosphodiester group that covalently couples adjacent nucleomonomers. Analogs or derivatives include phosphodiester analogs, e.g., phosphorothioate, phosphorodithioate, and P-ethyoxyphosphodiester, P-ethoxyphosphodiester, P-alkyloxyphosphotriester, methylphosphonate, phosphoramidates, thio-phosphoramidates, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47).

A non-nucleotidic linker or spacer sequence may be a peptide, a lipid, a polymer or an oligoethylene. Examples of linkers or spacers of the invention include HEG and PEG.

In an embodiment containing a liposomal core, the oligonucleotide shell may be anchored to the surface of the liposomal core through conjugation to one or a multiplicity of linker molecules including but not limited to: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In general, the oligonucleotide shell is anchored to the surface of the liposomal core through conjugation to one or a multiplicity of linker molecules including but not limited to: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In some embodiments at least 25, 50, 75, 100, 200, 300, 400, 500, 600, 700, 800, 900 or 1,000 oligonucleotides or any range combination thereof are on the exterior of the core. In some embodiments, 1-1,000, 5-1,000, 100-1,000, 500-1,000, 10-500, 50-250, or 50-300 oligonucleotides are present on the surface of the nanostructure. In another embodiment, 100 oligonucleotides are present on the surface of the nanostructure.

In general, the oligonucleotide shell is conjugated with a fluorophore or dye group, which is attached to either the 5′- or 3′-end of the oligonucleotide including, but not limited to: hydroxycoumarin, methoxycoumarin, Alexa fluor, coumarin, aminocoumarin, Cy2, FAM, Alexa fluor 405, Alexa fluor 488, Fluorescein FITC, Alexa Fluor 350, Alexa Fluor 405, Alexa fluor 430, Alexa fluor 532, HEX, Cy3, tetramethylrhodamine (TRITC), Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Alexa fluor 647, Cy5, Alexa fluor 660, Cy5.5, TruRed, Alexa fluor 680, Cy7, Cy7.5, Alexa Fluor 750, Oregon Green, Pacific Blue™, Pacific Orange™, Pacific Green™, BODIPY FL, and quantum dots. Other fluorophores and molecules that emit a detectable signal are encompassed by the present disclosure.

In some instances, the liposomal core may contain one or more fluorophores or dye molecules to further increase the signal emitted by the L-SNAs.

As used herein, the nanostructure is a construct having an average diameter on the order of nanometers (i.e., between about 1 nm and about 1 micrometer. For example, in some instances, the diameter of the nanoparticle is from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, about 1 nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention in the methods provided herein.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into diagnostic or research kits to facilitate their use in diagnostic or research applications. A kit may include one or more containers housing the components of the invention and instructions for use. Specifically, such kits may include one or more agents described herein, along with instructions describing the intended hybridization application and the proper administration of these agents.

As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of 1 products.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves etc.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use.

The kits, in one set of embodiments, may comprise a carrier means being compartmentalized to receive in close confinement one or more container means such as vials, tubes, and the like, each of the container means comprising one of the separate elements to be used in the method. For example, one of the containers may comprise a positive control for an assay. Additionally, the kit may include containers for other components, for example, buffers useful in the assay.

### EQUIVALENTS

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### EXAMPLES

### Example 1

A panel of oligonucleotides were 5′-modified with Cy5 and 3′-modified with a di-stearyl group and incorporated into small unilamellar vesicles (55 nm in diameter) composed of 1,2-dioleoyl-sn-glycero-3-phosphatidylcholine (DOPC) (FIG. 1) at a density of 100 oligonucleotides per vesicle. These L-SNAs FISH probes were tested for their ability to bind (β-Actin mRNA transcripts in fixed human cell lines for fluorescence in situ hybridization (FISH) imaging. Commercially available FISH probes (BioSearch Technologies) were used as a control.

### Methods

### A. Adherent Cell Fixation

Adherent cells are first grown on 12 mm round #1 Poly-L-Lysine coated coverslips in a 12-well cell culture plate for 24 hours prior to fixation. Growth medium is then aspirated, cells washed with 1 mL of 1× Phosphate Buffered Saline (PBS) RNAse free, and fixed for 10 minutes at room temperature in 1 mL of fixation buffer composed of 1:1:8 ratio of 37% formaldehyde solution, 10× PBS RNAse free, and nuclease free water respectively. Cells are then washed twice with 1 mL of 1× PBS RNAse free and permeabilized in 1 mL of 70% ethanol for at least 1 hour and up to a week at 2-8° C.

### B. Hybridization in Adherent Cells

70% ethanol is aspirated from the fixed cells, which are subsequently incubated at room temperature for 2-5 minutes in 1 mL of wash buffer composed of 1:1:8 ratio of 20× saline sodium citrate (SSC) buffer, formamide, and nuclease free water respectively. FISH probe solutions are diluted to a final working concentration in hybridization buffer composed of 1:1:1:7 ratio of dextran sulfate, formamide, 20×SSC, and nuclease free water respectively. 100 μL of working FISH probe solution is dispensed onto Parafilm in the humidifier chamber composed of 150 mm petri dishes lined with 3 layers of paper towels saturated with water and a dry layer of Parafilm on top. Coverslips are then placed cell side down onto 100 μL of working FISH probe solution in humidifier chamber and incubated in dark at 37° C. for 4-16 hours. Coverslips are then transferred into a new 12-well plate containing 1 mL of wash buffer and incubated in dark at 37° C. for 30 min. The wash buffer is then aspirated and a fresh 1 mL of wash buffer is added to coverslips which are then incubated at room temperature for 2-5 min. The coverslips are placed cells side down onto 7.5 μL of anti-fade mount with DAPI dispensed onto 1 mm thick microscope slides. Excess mount is wicked away and the coverslip edge sealed to the slide with clear nail polish.

### C. Fluorescence Analysis in Adherent Cells

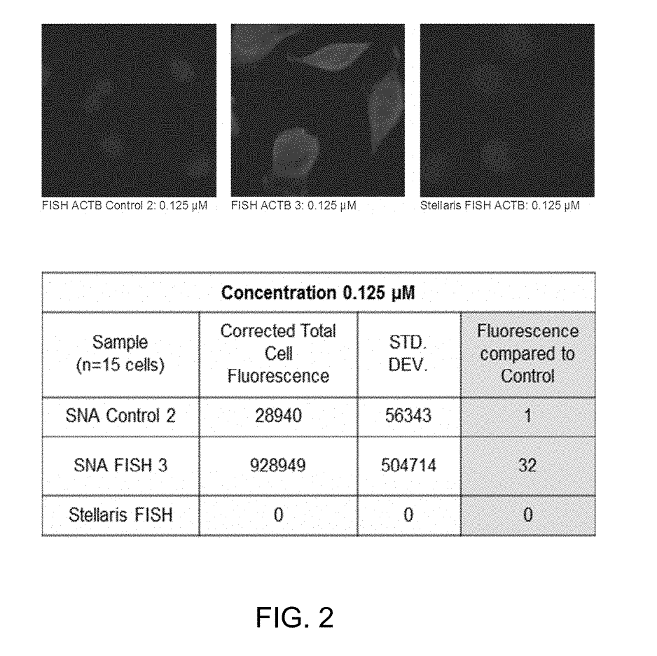

All cells are imaged with a 40× objective using a wide-field fluorescent microscope. Same scale settings are applied to all sample images taken. Using ImageJ, the total cell fluorescence is analyzed for each cell area in each sample and relative fluorescence compared between treatments (FIG. 2).

### Results

Fluorescence in situ hybridization (FISH) imaging is performed to investigate and compare the fluorescence intensity of L-SNA FISH probes to a commercially available FISH probe. To this end, adherent cells are fixed, permeabilized and incubated with control FISH L-SNAs, FISH L-SNAs that recognize (β-Actin, or a commercially available FISH probe that also recognizes (β-Actin. The fluorescence signals obtained by FISH are quantified by Image J. FISH imaging demonstrates L-SNAs containing oligonucleotides that recognize ACTB ((β-Actin) mRNA transcripts provide a higher level of fluorescence intensity (shown in red) relative to the commercially available Stellaris FISH probes that also recognize ACTB ((β-Actin) mRNA transcripts (FIG. 2). The results are confirmed by quantification of the signals by Image J. FISH L-SNAs have a 32-fold higher level of fluorescence relative to control FISH L-SNAs. The commercially available Stellaris FISH probes had lower fluorescence intensity than even control L-SNAs. Therefore, the results demonstrate that L-SNAs provide superior fluorescence intensity than a commercially available FISH probe.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of detecting a target nucleic acid in a sample by in situ hybridization, comprising:
incubating a sample with a liposomal spherical nucleic acid (L-SNA) probe and detecting probe hybridized to a nucleic acid target in the sample by visualization of a signal emitted from the L-SNA probe.

2. The method of claim 1, wherein the in situ hybridization is fluorescence in situ hybridization (FISH).

3. The method of claim 1, wherein the sample is fixed.

4. The method of claim 1, wherein the L-SNA probe is comprised of a liposomal core and an oligonucleotide shell or 2-500 oligonucleotides surrounding the liposomal core and linked to a dye molecule.

5. The method of claim 4, wherein the dye molecule is attached to either the 5′- or 3′-end of the oligonucleotides.

6. The method of claim 4, wherein the dye molecule is a fluorophore.

7. The method of claim 4, wherein the oligonucleotides are anchored to the liposomal core and point radially outwards from the core.

8. The method of claim 4, wherein the oligonucleotides are attached to a hydrophobic anchor group at the 5′- or 3′-end to insert the oligonucleotides into the liposomal core and/or anchor the oligonucleotides to the liposomal core.

9. The method of claim 8, wherein the hydrophobic anchor group is a lipid.

10. The method of claim 9, wherein the lipid is a di-stearyl group.

11. The method of claim 4, wherein the liposomal core comprises 1,2-dioleoyl-sn-glycero-3-phosphatidylcholine (DOPC).

12. The method of claim 4, wherein the L-SNA is comprised of about 100 oligonucleotides.

13. The method of claim 4, wherein the L-SNA is comprised of 80-120 oligonucleotides.

14. The method of claim 4, wherein the L-SNA is comprised of 50-200 oligonucleotides.

15. The method of claim 1, wherein the signal depicting the nucleic acid target is visualized by fluorescence microscopy.

16. The method of claim 1, wherein the sample is a cell or a tissue.

17. The method of claim 16, wherein the sample is a low abundance cell.

18. The method of claim 1, wherein the method is used to diagnose a disease.

19. The method of claim 1, wherein the nucleic acid target is RNA.

20. The method of claim 1, wherein the nucleic acid target is DNA.

21. The method of claim 1, wherein the oligonucleotide is DNA

22. The method of claim 1, wherein the oligonucleotide is RNA.

23. The method of claim 1, further comprising:
(a) attaching the sample, wherein the sample is a cell, to a solid surface;
(b) permeabilizing the cell in 70% ethanol for at least 1 hour and up to a week at 2-8° C.;
(c) incubating the cell with L-SNA probes in hybridization buffer composed of 1:1:1:7 ratio of dextran sulfate, formamide, 20×SSC, and nuclease free water in the dark at 37° C. for 4-16 hours;
(d) counterstaining the cell with a nuclear stain; and
(e) detecting a fluorescent signal.

24. A kit, comprising:
a lipid for producing a liposome,
a fluorescent dye,
one or more reagents for performing a FISH assay, and
instructions for performing a FISH assay using a L-SNA probe.

25. The kit of claim 24, further comprising a set of oligonucleotides for preparing a L-SNA probe.

26. The kit of claim 24, wherein the fluorescent dye is linked to the oligonucleotides.

27. The kit of claim 26, wherein the fluorescent dye is attached to either the 5′- or 3′-end of the oligonucleotide.

28. The kit of claim 27, wherein the fluorescent dye is a fluorophore.

29. The kit of claim 28, wherein the fluorophore is Cy5.

30. The kit of claim 25, wherein the oligonucleotides have a hydrophobic or lipid anchor group at a 5′ or 3′ end.

31. The kit of claim 30, wherein the hydrophobic or lipid anchor group is a di-stearyl group.

32. The kit of claim 24, wherein the lipid is 1,2-dioleoyl-sn-glycero-3-phosphatidylcholine (DOPC).

33. The kit of claim 25, wherein the oligonucleotide is DNA

34. The kit of claim 25, wherein the oligonucleotide is RNA.
